(19) United States
(12) Patent Application Publication
(10) Pub. No.: US 2025/0352483 A1
(43) Pub. Date: Nov. 20, 2025

(54) Biomimetic Drug Delivery Particles

(71) Applicant: Naval Information Warfare Center Pacific, San Diego, CA (US)

(72) Inventor: Mario Malfavon, Spring Valley, CA (US)

(73) Assignee: THE UNITED STATES OF AMERICA, AS REPRESENTED BY THE SECRETARY OF THE NAVY, San Diego, CA (US)

(21) Appl. No.: 18/663,197

(22) Filed: May 14, 2024

(51) Int. Cl. A61K 9/50; A61K 45/06; A61K 47/62; A61K 47/69

(52) U.S. Cl. CPC A61K 9/5031; A61K 9/5089; A61K 45/06; A61K 47/62; A61K 47/6927

(57) ABSTRACT

Each biomimetic drug delivery particle includes a microparticle and one or more types of endogenous peptides. The microparticle is a biodegradable copolymer. The one or more types of endogenous peptides are bonded to a surface of the microparticle in an amount ranging from about 15% to about 50% coverage of the surface of the microparticle.

### STATEMENT REGARDING FEDERALLY SPONSORED RESEARCH OR DEVELOPMENT

The invention described herein may be manufactured and used by or for the government of the United States of America for governmental purposes without the payment of any royalties thereon or therefor. Licensing and technical inquiries may be directed to the Office of Research and Technical Applications, Naval Information Warfare Center Pacific, Code 72120, San Diego, CA, 92152; (619) 553-5118; NIWC_Pacific_T2@us.navy.mil. Reference Navy Case Number 211472.

### BACKGROUND

Ophthalmic diseases are diseases or disorders related to the eyes. Diagnosis of ophthalmic diseases in mammals require extensive, time consuming, and invasive procedures. For some mammals, such as dolphins, the cause and mechanism of progression is not well understood. Some ophthalmic diseases that are treated, often unsuccessfully, require surgical intervention to treat, which can be risky, challenging, and laborious. Other current treatment methods aside from surgery include sub-corneal injections, which can have more success, but are difficult to administer to some mammals, especially marine mammals.

### DESCRIPTION OF THE DRAWINGS

Features and advantages of examples of the present disclosure will be apparent by reference to the following detailed description and drawings, in which like reference numerals correspond to similar, but in some instances, not identical, components. Reference numerals or features having a previously described function may or may not be described in connection with other drawings in which they appear.

FIG. 1 is an example of the chemical synthesis of the biomimetic drug delivery particles described herein; and

FIG. 2A-2B is an example of a diagram showing non-biomimetic drug delivery particles and a diagram showing the biomimetic drug deliver particles described herein, respectively.

### DETAILED DESCRIPTION

Ophthalmic disease is one of the most common diseases affecting dolphins. The specific cause and mechanism of progression is not well understood. Diagnosis and treatment typically require extensive, time-consuming, and invasive procedures. Treatment of disease with topical drugs is inexpensive, but often ineffective due to thick dolphin tear film with unique chemistry preventing medication from reaching the corneal surface before being flushed from the eye surface in water. Sub-corneal injection requires greater labor and is significantly more invasive, but can obtain better treatment outcomes. However, due to a lack of efficient, non-invasive methods of treatment many ophthalmic diseases have poor outcomes in dolphins.

A biomimetic drug deliver particle is described herein that provides a significantly less invasive drug delivery method compared to the current treatment methods. The drug delivery particle includes bonded endogenous peptides that are derived from proteins found within mammals. The endogenous peptides can be synthetically produced and bonded to the drug delivery particle to allow the drug delivery particle to absorb into the tear film within the eye. Not only does this allow more effective treatment of ophthalmic diseases, but the treatment is less invasive, less time-consuming, and less expensive compared to traditional treatment methods.

Each biomimetic drug delivery particle herein includes a microparticle and one or more types of endogenous peptides. The microparticle is a biodegradable copolymer. The one or more types of endogenous peptides are bonded to a surface of the microparticle in an amount ranging from about 15% to about 50% coverage of the surface of the microparticle.

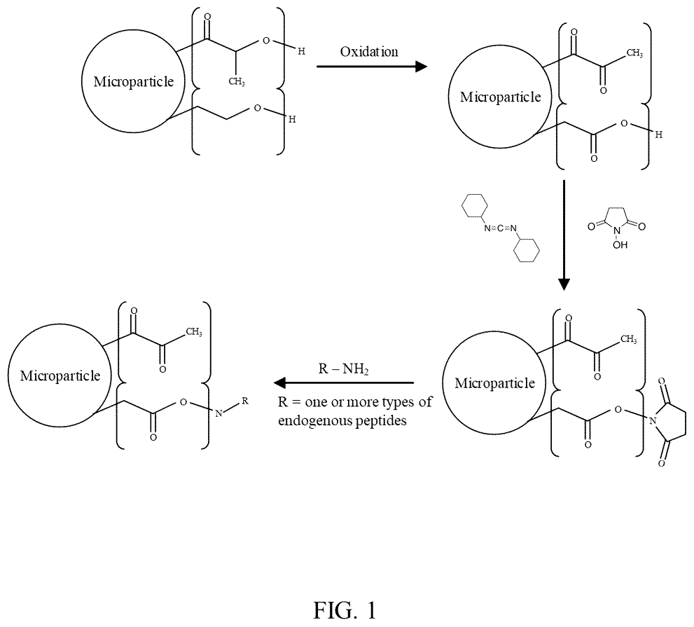

Referring now to FIG. 1, an example of a method for synthesizing the biomimetic drug delivery particle is shown. In this example, the method involves oxidizing the surface of biodegradable copolymer microparticles, thereby bonding carboxylic acid groups to the biodegradable copolymer microparticles. In this example, the biodegradable copolymer microparticles are poly (lactic-co-glycolic acid)-poly (glycolic acid) microparticles or a poly (lactic-co-glycolic acid)-polyethylene glycol microparticles. In an example, the biodegradable copolymer microparticle has a ratio of poly (glycolic acid) or polyethylene glycol to poly (lactic-co-glycolic acid) of about 15% to about 50% of the microparticle. In another example, the biodegradable copolymer microparticle has a ratio of poly (glycolic acid) or polyethylene glycol to poly (lactic-co-glycolic acid) of about 15% to about 33% of the microparticle. In some examples, the biodegradable copolymer microparticles include an ocular drug contained within the microparticle. The ocular drug may be selected from a drug that treats a viral infection, a bacterial infection, a fungal infection, or a combination thereof.

Any oxidizing agent may be used that bonds the carboxylic acid groups to the biodegradable copolymer microparticles. Some examples include KMnO4, H2O2, H2SO4, O2, or a combination thereof.

Referring back to FIG. 1, the method involves modifying carboxylic acid groups with one or more imides, thereby bonding imide leaving groups to the biodegradable copolymer microparticles. Any imide that provides an imide leaving group on the biodegradable copolymer microparticles may be used. In an example, the one or more imides may be N,N′-dicyclohexylcarbodiimide, N-hydroxysuccinimide, or a combination thereof. As shown in FIG. 1, a combination of N,N′-dicyclohexylcarbodiimide and N-hydroxysuccinimide is used to add the imide leaving groups to the biodegradable copolymer microparticles.

Referring back to FIG. 1, the method involves modifying the imide leaving group with one or more types of endogenous peptides, thereby forming the biomimetic drug delivery particle including the biodegradable copolymer microparticle with one or more types of endogenous peptides bonded to a surface of the biodegradable copolymer microparticle. In an example, the one or more types of endogenous peptides may be one or more types of any endogenous protein found in mammals. In another example, the one or more types of endogenous peptides may be one or more types of any protein found in dolphins. In another example, the one or more types of endogenous peptides may be derived from one or more proteins selected from a group consisting of a keratin type II cytoskeletal 6A-like isoform X3 protein, an IgGFc-binding protein, a keratin type I cytoskeletal 14 protein, lysozyme, a tenascin isoform X5 protein, a polymeric immunoglobulin receptor protein, a serum albumin isoform X2 protein, a mucin-19 protein, a keratin, type I cytoskeletal 13-like protein, an involucrin protein, protein LEG1 homolog, aldehyde dehydrogenase, a C3-beta-c protein, an alpha-1-antitrypsin-like protein, deoxyribonuclease, an immunoglobulin lambda-1 light chain-like isoform X2 protein, a desmoplakin isoform X1 protein, a lactotransferrin protein, an actin cytoplasmic 1 protein, an immunoglobulin heavy constant alpha 2 protein, a keratin, type II cytoskeletal 5 protein, a thrombospondin-1 protein, and combinations thereof. For example, one or more types of endogenous peptides may be derived from a keratin type II cytoskeletal 6A-like isoform X3 protein individually or in combination with another protein, such as an IgGFc-binding protein.

The one or more types of endogenous peptides may be derived from any protein listed individually or in any combination. In FIG. 1, the endogenous peptide includes an amine group that allows the one or more types of endogenous peptides to bond to the carboxylic acid created after oxidation of poly (glycolic acid) or polyethylene glycol. However, the one or more types of endogenous peptides do not bond with the ketone created after oxidation of the poly(lactic-co-glycolic acid).

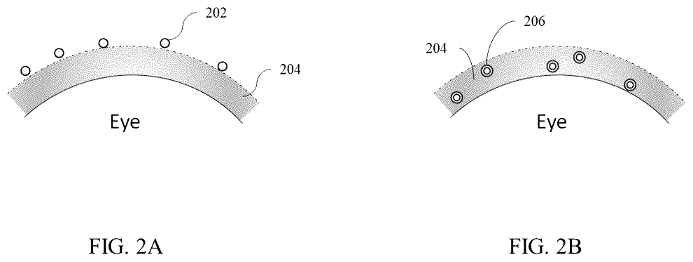

Referring now to FIG. 2A-2B, an example of the biodegradable copolymer microparticles without the one or more types of endogenous peptides bonded to the surfaces of the biodegradable copolymer microparticles and the biodegradable copolymer microparticles with the one or more types of endogenous peptides bonded to the surfaces of the biodegradable copolymer microparticles, respectively. The shading and hatching patterning is for illustrative purposes only to aid in viewing and should not be construed as being limiting or directed to a particular material or materials. In FIG. 2A, the biodegradable copolymer microparticles 202 are unable to enter the tear film 204 of the eye due to the lack of any endogenous peptides. The lack of endogenous peptides causes on the microparticles causes a mismatch in chemistry that prevents the microparticles from passing into the tear film 204.

In contrast, in FIG. 2B, the biomimetic drug delivery particles 206 include the microparticle and one or more types of endogenous peptides. The microparticle is a biodegradable copolymer microparticle. The one or more types of endogenous peptides allow the biomimetic drug delivery particles 206 to enter into the tear film 204 due to the chemical compatibility with the eye.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein.

As used herein, a plurality of items, structural elements, compositional elements, and/or materials may be presented in a common list for convenience. However, these lists should be construed as though each member of the list is individually identified as a separate and unique member. Thus, no individual member of a list should be construed as a de facto equivalent of any other member of the same list merely based on their presentation in a common group without indications to the contrary.

Unless otherwise stated, any feature described herein can be combined with any aspect or any other feature described herein.

Reference throughout the specification to “one example”, “another example”, “an example”, means that a particular element (e.g., feature, structure, and/or characteristic) described in connection with the example is included in at least one example described herein, and may or may not be present in other examples. In addition, the described elements for any example may be combined in any suitable manner in the various examples unless the context clearly dictates otherwise.

The ranges provided herein include the stated range and any value or sub-range within the stated range. For example, a range from about 0.1 to about 20 should be interpreted to include not only the explicitly recited limits of from about 0.1 to about 20, but also to include individual values, such as 3, 7, 13.5, etc., and sub-ranges, such as from about 5 to about 15, etc.

In describing and claiming the examples disclosed herein, the singular forms “a”, “an”, and “the” include plural referents unless the context clearly dictates otherwise.

What is claimed is:

1. Biomimetic drug delivery particles, each particle comprising:
a microparticle, wherein the microparticle is a biodegradable copolymer microparticle;
one or more types of endogenous peptides, wherein the one or more types of endogenous peptides are bonded to a surface of the microparticle in an amount ranging from about 15% to about 50% coverage of the surface of the microparticle.

2. The biomimetic drug delivery particles of claim 1, wherein the microparticle is a poly (lactic-co-glycolic acid)-poly (glycolic acid) microparticle or a poly (lactic-co-glycolic acid)-polyethylene glycol microparticle.

3. The biomimetic drug delivery particles of claim 2, wherein the microparticle has a ratio of poly (glycolic acid) or polyethylene glycol to poly (lactic-co-glycolic acid) of about 15% to about 50% of the microparticle.

4. The biomimetic drug delivery particles of claim 1, wherein the one or more types of endogenous peptides are derived from one or more proteins selected from a group consisting of a keratin type II cytoskeletal 6A-like isoform X3 protein, an IgGFc-binding protein, a keratin type I cytoskeletal 14 protein, lysozyme, a tenascin isoform X5 protein, a polymeric immunoglobulin receptor protein, a serum albumin isoform X2 protein, a mucin-19 protein, a keratin, type I cytoskeletal 13-like protein, an involucrin protein, protein LEG1 homolog, aldehyde dehydrogenase, a C3-beta-c protein, an alpha-1-antitrypsin-like protein, deoxyribonuclease, an immunoglobulin lambda-1 light chain-like isoform X2 protein, a desmoplakin isoform X1 protein, a lactotransferrin protein, an actin cytoplasmic 1 protein, an immunoglobulin heavy constant alpha 2 protein, a keratin, type II cytoskeletal 5 protein, a thrombospondin-1 protein, and combinations thereof.

5. The biomimetic drug delivery particles of claim 1, wherein the microparticle includes an ocular drug contained within the microparticle, the ocular drug selected from a drug that treats a viral infection, a bacterial infection, a fungal infection, or a combination thereof.

6. The biomimetic drug delivery particles of claim 1, wherein the one or more types of endogenous peptides are from mammals.

7. The biomimetic drug delivery particles of claim 1, wherein the one or more types of endogenous peptides are from dolphins.

8. A method of making biomimetic drug delivery particles, comprising:
oxidizing the surface of biodegradable copolymer microparticles, thereby bonding carboxylic acid groups to the biodegradable copolymer microparticles;
modifying the carboxylic acid groups with one or more imides, thereby bonding imide leaving groups to the biodegradable copolymer microparticles; and
modifying the imide leaving group with one or more types of endogenous peptides, thereby forming the biomimetic drug delivery particle including the biodegradable copolymer microparticle with the one or more types of endogenous peptides bonded to a surface of the biodegradable copolymer microparticle.

9. The method of claim 8, wherein the biodegradable copolymer microparticles are poly (lactic-co-glycolic acid)-poly (glycolic acid) microparticles or a poly (lactic-co-glycolic acid)-polyethylene glycol microparticles.

10. The method of claim 9, wherein the biodegradable copolymer microparticle has a ratio of poly (glycolic acid) or polyethylene glycol to poly (lactic-co-glycolic acid) of about 15% to about 50% of the microparticle.

11. The method of claim 8, wherein the one or more types of endogenous peptides are derived from one or more proteins selected from a group consisting of a keratin type II cytoskeletal 6A-like isoform X3 protein, an IgGFc-binding protein, a keratin type I cytoskeletal 14 protein, lysozyme, a tenascin isoform X5 protein, a polymeric immunoglobulin receptor protein, a serum albumin isoform X2 protein, a mucin-19 protein, a keratin, type I cytoskeletal 13-like protein, an involucrin protein, protein LEG1 homolog, aldehyde dehydrogenase, a C3-beta-c protein, an alpha-1-antitrypsin-like protein, deoxyribonuclease, an immunoglobulin lambda-1 light chain-like isoform X2 protein, a desmoplakin isoform X1 protein, a lactotransferrin protein, an actin cytoplasmic 1 protein, an immunoglobulin heavy constant alpha 2 protein, a keratin, type II cytoskeletal 5 protein, a thrombospondin-1 protein, and combinations thereof.

12. The method of claim 8, wherein the biodegradable copolymer microparticles include an ocular drug contained within the microparticle, the ocular drug selected from a drug that treats a viral infection, a bacterial infection, a fungal infection, or a combination thereof.

13. The method of claim 8, wherein oxidizing the surface of biodegradable copolymer microparticles is H₂CrO₄, KMnO₄, H₂O₂, H₂SO₄, O₂, or a combination thereof.

14. The method of claim 8, wherein the imide is N,N-dicyclohexylcarbodiimide, N-hydroxysuccinimide, or a combination thereof.
